(19) United States
(12) Patent Application Publication
(10) Pub. No.: US 2022/0160273 A1
(43) Pub. Date: May 26, 2022

(54) PORTABLE AUTONOMOUS VENIPUNCTURE DEVICE

(71) Applicant: Alireza Bagheri Rajeoni, Akron, OH (US)

(72) Inventor: Alireza Bagheri Rajeoni, Akron, OH (US)

(21) Appl. No.: 17/101,076

(22) Filed: Nov. 23, 2020

(51) Int. Cl. A61B 5/15; A61B 8/08; A61B 5/00; A61M 5/20; A61M 5/315; A61N 1/39

(52) U.S. Cl. CPC A61B 5/150748; A61M 2205/13; A61B 5/150954; A61B 8/085; A61B 5/0077; A61B 5/150236; A61M 5/20; A61M 5/31586; A61N 1/3904; A61B 2560/0456; A61M 2205/3375; A61M 2205/3313; A61M 2209/088; A61M 2205/50; A61M 2205/103; A61M 2005/206; A61M 2205/054; A61B 5/15003

(57) ABSTRACT

A portable autonomous venipuncture device to be secured to a limb of a patient comprising a limb-mountable base configured to be mounted to the limb of a patient, a venipuncture tool holder carried on said base, a venipuncture tool carried by said venipuncture tool holder and having an insertion tip; an ultrasonic device carried on said base; and a processor. The ultrasonic device provides an ultrasonic image of inside of the limb of the patient to provide location data on the position and depth of a vein in the limb. The processor is configured to use the location data to move the insertion tip of the venipuncture tool into the vein of the patient.

### CROSS-REFERENCE TO RELATED APPLICATIONS

The present application claims the benefit of U.S. Provisional Patent Application No. 62/938,421, filed on Nov. 21, 2019 which is incorporated herein in its entirety.

### FIELD OF THE INVENTION

The present invention generally relates to a portable autonomous venipuncture device that provides a portable means to supply venous access for blood drawings or drug injections. Particularly, the present invention relates to a portable autonomous venipuncture device used in conjunction with an automated external defibrillator (AED) device to insert adrenalin if needed after the delivery of a shock from the AED device. More particularly, the present invention relates to an autonomous venipuncture device that utilizes ultrasound and infrared light to determine proper vein location and depth detection of the vein prior to insertion.

### BACKGROUND OF THE INVENTION

Every year 790,000 people in the United States have a heart attack. Among them, 510,000 are having their first heart attack. Around 610,000 people in the United States die every year due to heart disease and half of this group die outside of a hospital setting. Sudden cardiac arrest is not a heart attack but can occur during a heart attack. Heart attacks occur when there is a blockage in one or more of the arteries leading to the heart, which prevents the heart from receiving enough oxygen-rich blood. If the oxygen in the blood cannot reach the muscles of the heart, then the heart becomes damaged.

In contrast, sudden cardiac arrest occurs when the electrical system to the heart malfunctions and suddenly becomes irregular. The heart then starts to beat dangerously fast and the ventricles may begin to flutter or quiver and then blood is not delivered to the body. In the first few minutes, the greatest concern is that blood flow to the brain will be reduced to a point that a person will lose consciousness. Death will soon follow unless emergency treatment is begun.

In the past few decades, there have been vital inventions that have hugely reduced death caused by heart disease. Automated external defibrillators (AED) is one of those inventions. An AED can detect arrhythmia and can treat it through defibrillation. An AED is easy to use and is designed to be used for un-trained users and are typically placed in public places. Yet, there are some instances that arrhythmia is not shockable such as Bradycardia or it is shock refractory. In these instances, it is recommended by the American Heart Association to insert a drug to assist.

Nearly 250 million intravenous (IV) catheterizations take place in the United States annually, and 28% of those insertions fail on the first attempt in normal adults. The failure rate for children is as high as 54%. At least 27% of patients require 3 attempts or more to obtain a successful insertion. Beyond producing pain and disfiguration, failed sticks can result in permanent injury such as venous scarring and nerve damage. In patients with chronic illnesses that require repeated insertions, the veins can scar to an extent that insertions are impossible in hands or arms. Furthermore, venous access is currently only available in a hospital setting or via a trained professional utilizing IV access which limits the drug therapy of outpatients to oral and/or subcutaneous drug therapy. Exceptions to the norm being Peripherally Inserted Central Lines of Midlines which are both not only very expensive, but both are very invasive and could cause serious infections if not properly inserted.

Diagnostic blood testing is the most common medical routine performed in the world, and in many ways, forms the cornerstone of modern medicine. In the United States, blood tests are performed 2 billion times each year and influence 80% of medical decisions made in hospital and primary care settings. However, blood draw success rates depend heavily on practitioner skill and patient physiology.

As discussed above, intravenous needle and cannula insertion are common procedures within modern medicine. They are considered essential components of both drug delivery and blood sampling for diagnostic and treatment purposes. Even though intravenous needle insertion in forearm veins is one of the most practiced medical procedures, it is notorious for being an unmastered technique. Many patients are poked several times before the needle is successfully inserted and there is often great variability in adeptness among medical personal in inserting the needles. In the past decade, robotic technology has slowly made its push into the medical field. Advanced surgical robots have become technological icons in today's hospitals. However, the use of robotics for everyday medicine remains a major challenge due to the cost and size of current systems. Accordingly, there is a need to automate needle insertion to lessen the dependence on skilled technicians. Furthermore, there is a need to automate needle insertion when skilled technicians are not available, such as when an AED device is being used in the public by a non-skilled person.

### SUMMARY OF THE INVENTION

An embodiment of the present invention provides a portable autonomous venipuncture device to be secured to a limb of a patient comprising: a limb-mountable base configured to be mounted to the limb of a patient; a venipuncture tool holder carried on said base a venipuncture tool carried by said venipuncture tool holder and having an insertion tip; an ultrasonic device carried on said base; and a processor, wherein said ultrasonic device is configured to provide an ultrasonic image of inside of the limb of the patient to provide location data on the position and depth of a vein in the limb, and wherein said processor is configured to use the location data to move the insertion tip of the venipuncture tool into the vein of the patient

Another embodiment of the present invention provides a portable autonomous venipuncture device as in any embodiment above, further comprising a first motor to control movement of said venipuncture tool holder along an X-axis and a second motor to control movement of said venipuncture tool holder along a Y-axis.

Another embodiment of the present invention provides a portable autonomous venipuncture device as in any embodiment above, wherein further comprising an X-linear motion stage, an X-axis screw, a Y-linear motion stage, and a Y-axis rail.

Another embodiment of the present invention provides a portable autonomous venipuncture device as in any embodiment above, wherein said processor directs the first motor to move the X-linear motion stage along the X-axis screw to place said venipuncture tool holder into an X-axis position; and wherein said processor directs the second motor to move the Y-linear motion stage along the Y-axis rail to place said venipuncture tool holder into a Y-axis position.

Another embodiment of the present invention provides a portable autonomous venipuncture device as in any embodiment above, further comprising an infrared camera configured to provide an infrared image of the limb of the patient to provide additional location data on the position of the vein in the limb, and wherein said processor is configured to use the location data from the ultrasonic device said additional location data from said infrared camera to move the insertion tip of the venipuncture tool into the vein of the patient.

Another embodiment of the present invention provides a portable autonomous venipuncture device as in any embodiment above, wherein said base further comprises a front pair of apertures, a rear set of apertures, and an insertion site aperture wherein said insertion site aperture gives said venipuncture tool access to said limb through said base.

Another embodiment of the present invention provides a portable autonomous venipuncture device as in any embodiment above, further comprising a front strap placed through said front pair of apertures and a rear strap placed through said rear set of apertures.

Another embodiment of the present invention provides a portable autonomous venipuncture device as in any embodiment above, further including a drug delivery system carried on said base, wherein said drug delivery system comprises: a drug/fluid storage device carried on the limb-mountable base; a pipe connected to said drug/fluid storage device; a syringe motor carried on the limb-mountable base; a syringe screw; a plunger plate connected to the syringe screw; and a syringe connected to the plunger plate and carried by the drug/fluid storage device; and wherein the syringe motor controls movement of the plunger plate along the syringe screw to operate the syringe to deliver a drug carried within the drug/fluid storage device through the pipe and into the limb or to operate the syringe to draw blood from the limb and deliver the drawn blood through the pipe to the drug/fluid storage device.

Another embodiment of the present invention provides a portable autonomous venipuncture device as in any embodiment above, wherein the venipuncture tool holder further includes a pipe access port, and wherein a first end of the pipe is connected to the drug/fluid storage device and a second end of the pipe is connected to the pipe access port.

An embodiment of the present invention provides a medical assistance and drug delivery device comprising a medical assistance device and an autonomous venipuncture device including a processor, wherein the medical assistance device provides medical assistance to a patient while also producing a medical data set, wherein the processor assesses the medical data set to determine if an injection-treatable condition is present, and if the injection-treatable condition is present, the autonomous venipuncture device delivers an injection.

An embodiment of the present invention provides a method of treating an arrhythmia of a patient comprising the steps of: placing electrode pads of an automated external defibrillator (AED) device on a patient's chest; placing an autonomous venipuncture device on a limb of the patient, the autonomous venipuncture device having a venipuncture tool having an insertion tip; reading by the AED device of a heart rhythm of the patient to determine if said rhythm is a shockable arrhythmia; optionally delivering at least one shock to the chest of the patient through the pair of electrode pads; determining if drug assistance is needed to treat the heart rhythm; determining an insertion position on the limb utilizing the autonomous venipuncture device; inserting, if drug assistance was determined to be needed, the insertion tip of the venipuncture tool into the insertion position on the limb of the patient; and delivering drug assistance to the patient through the venipuncture tool.

Another embodiment of the present invention provides a method of treating an arrhythmia of a patient as in any embodiment above, wherein the drug assistance can be delivered through the venipuncture tool directly by the autonomous venipuncture device or by a trained professional.

Another embodiment of the present invention provides a method of treating an arrhythmia of a patient as in any embodiment above, wherein the drug assistance is delivered directly by the autonomous venipuncture device by a syringe motor of the autonomous venipuncture device operating a syringe connected to a plunger plate of the autonomous venipuncture device along a syringe screw of the autonomous venipuncture device to deliver a drug carried within a drug/fluid storage device of the autonomous venipuncture device through a pipe that connects to the venipuncture tool.

Another embodiment of the present invention provides a method of treating an arrhythmia of a patient as in any embodiment above, wherein the determining of the insertion position is conducted by an ultrasonic device of the autonomous venipuncture device taking an ultrasonic image of inside of the limb of the patient to provide location data on the position and depth of a vein in the limb, and wherein said processor is configured to use the location data to move the insertion tip of the venipuncture tool into the vein of the patient.

Another embodiment of the present invention provides a method of treating an arrhythmia of a patient as in any embodiment above, wherein a first motor of the autonomous venipuncture device controls the positioning of said venipuncture tool along an X-axis and a second motor of the autonomous venipuncture device controls positioning of said venipuncture tool along a Y-axis.

Another embodiment of the present invention provides a method of treating an arrhythmia of a patient as in any embodiment above, wherein the determining of the insertion position is further conducted by an infrared camera of the autonomous venipuncture device which takes an infrared image of the limb of the patient to provide additional data on the position of the vein in the limb, and wherein said processor takes said data from said ultrasonic device and said additional data from said infrared camera to move the insertion tip of the venipuncture tool into the insertion position.

### BRIEF DESCRIPTION OF THE DRAWINGS

FIG. 1 shows an embodiment of the medical assistance and drug delivery device of the present invention;

FIG. 2 shows a flow chart on how to use the medical assistance and drug delivery device of the present invention;

FIG. 3 shows a first embodiment of a portable autonomous venipuncture device of the present invention; and

FIG. 4 shows a second embodiment of a portable autonomous venipuncture device of the present invention.

### DETAILED DESCRIPTION OF ILLUSTRATIVE EMBODIMENTS

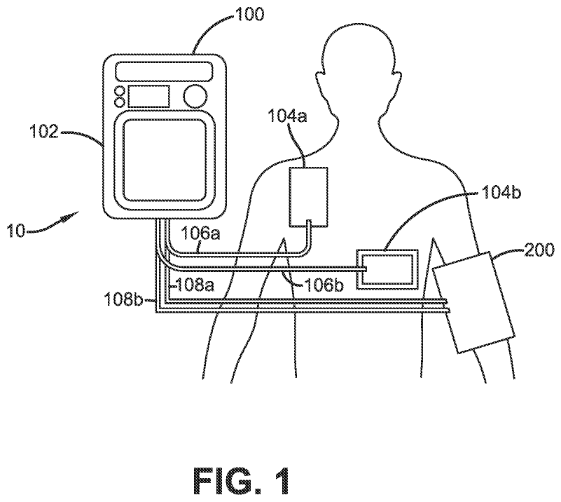

FIG. 1 shows a first embodiment of the present invention which shows a medical assistance and drug delivery device 10 which includes the combination of a medical assistance device 100 and a portable autonomous venipuncture device 200. In addition to the medical assistance device 100 and an autonomous venipuncture device 200, device 10 also includes a processor. The medical assistance device 100 provides medical assistance to a patient and while providing the medical assistance, device 100 also produces a medical data set. The processor will assess the medical data set produced by the medical assistance device 100 to determine if an injection-treatable condition is present within the patient. If there is an injection-treatable condition present, the autonomous venipuncture device 200 will deliver an injection to the patient.

In one or more embodiments, the medical assistance device 100 is any device that can perform one or more of the following monitoring activities: monitoring the heart rhythm of a patient, monitoring the glucose levels of the patient, testing for pregnancy/ovulation in the patient, monitoring of prothrombin time, monitoring for fecal occult blood, testing for the presence of drugs of abuse in the patient, detection of bacterial infections in the patient, and or the monitoring of the cholesterol levels of a patient.

In one or more embodiments, the medical assistance device 100 is selected from the group consisting of an automated external defibrillator (AED) device or any other wearable device that utilizes sensors to monitor a physiological status of a patient. An automated external defibrillator (AED) device 100 is shown in FIG. 1 and will be referenced to for further discussions involving device 10. Device 10 does not rely on a specific AED device 100, so any known AED device 100 can be utilized within device 10. Similarly, device 10 does not rely on a specific autonomous venipuncture device 200, so any autonomous venipuncture device 200 can be utilized within device 10, although a specific autonomous venipuncture device 200 will be described in detail later.

AED device 100 comprises a case 102 for housing a power supply (not shown) that is electrically connected to a circuit (not shown) for generating a defibrillation pulse. The circuit is electrically connected to a pair of electrode pads 104a and 104b that are applied to a patient to deliver the defibrillation pulse and to read the electrocardiogram (ECG) signals of the patient. The pair of electrodes are connected to case 102 through a pair of wires 106a and 106b. Autonomous venipuncture device 200 is also connected to case 102 through a pair of wires 108a and 108b.

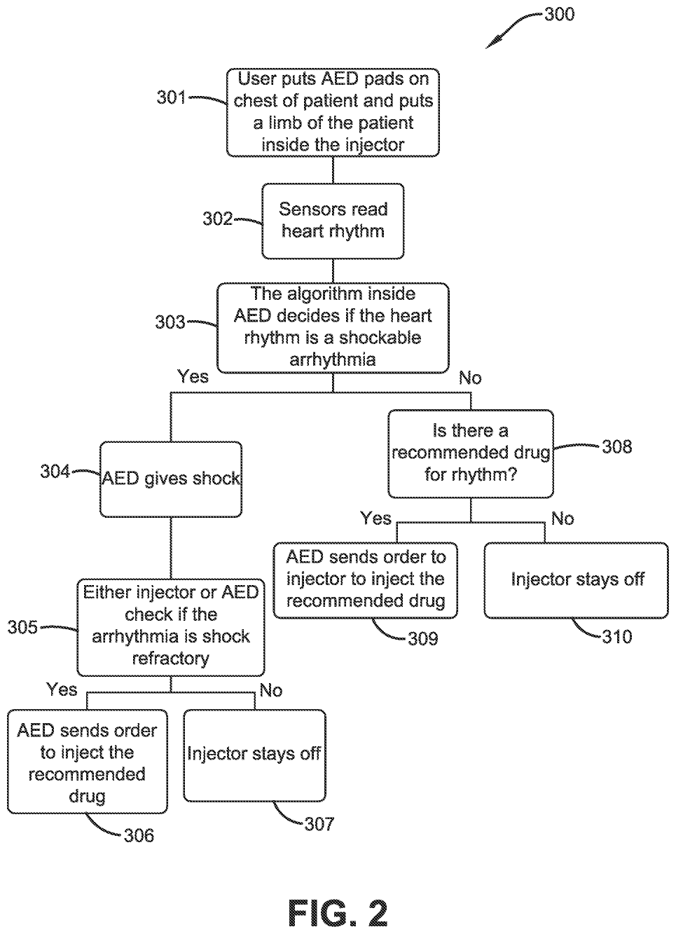

A methodology 300 to use device 10 is shown in the chart of FIG. 2. At a first step 301 of method 300, the user first places the pair of electrode pads 104a and 104b on the chest of the patient while placing the autonomous venipuncture device 200 in place around a limb of the patient. At a second step 302, electrode pads 104a and 104b read the heart rhythm of the patient. At a third step 303, the AED device 100 decides if the heart rhythm of the patient is a shockable arrhythmia. Next, if the heart rhythm of the patient is a shockable arrhythmia, method 300 moves on to step 304 wherein the AED device 100 delivers a shock. The delivery of a shock at step 304 can be repeated as many times as necessary to treat the patient.

After the appropriate number of shocks have been delivered to the patient, then method 300 moves onto step 305, wherein the AED device 100 or the autonomous venipuncture device 200 checks to see if the arrhythmia is shock refractory. Next, if the arrhythmia is shock refractory, method 300 moves onto step 306, wherein AED device 100 sends an order to the autonomous venipuncture device 200 to insert a venipuncture tool into the limb of the patient. With a venipuncture tool in place, a first responder will then have easy and accurately placed access to the patient in order to deliver a drug, or, if the autonomous venipuncture device 200 has the technology in order to deliver a drug itself, then the autonomous venipuncture device 200 can insert a drug. If the arrhythmia is not shock refractory, method 300 moves on to step 307, wherein the autonomous venipuncture device 200 stays off.

If the heart rhythm of the patient is not a shockable arrhythmia, method 300 would not move to step 304, and would instead move to step 308 wherein a determination is made as to whether there is a recommendation to deliver a drug to assist with treating the heart rhythm of the patient. Next, if a determination is made that drug assistance is needed, method 300 moves to step 309, wherein AED device 100 sends an order to the autonomous venipuncture device 200 to insert a venipuncture tool into the limb of the patient. With a venipuncture tool in place, a first respondent will then have easy and accurate access to the patient in order to deliver a drug, or, if the autonomous venipuncture device 200 has the technology in order to deliver a drug itself, then the autonomous venipuncture device 200 can deliver a drug. If there is not a recommendation to deliver a drug, method 300 moves on to step 310, wherein the autonomous venipuncture device 200 stays off.

In one or more embodiments of method 300 as discussed above, if the autonomous venipuncture device 200 has the technology in order to deliver a drug itself, the drug utilized by autonomous venipuncture device 200 can be selected from any recommended or prescribed drug. In one or more embodiments, if the autonomous venipuncture device 200 does not have the technology in order to deliver a drug, then device 200 will still be able to provide IV access for a trained professional to deliver drugs to the patient.

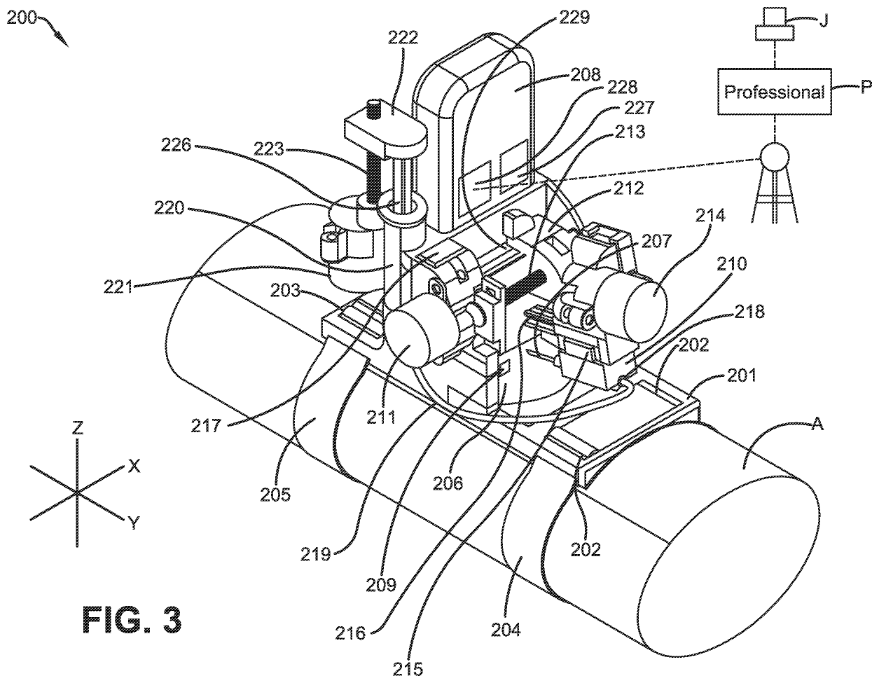

One embodiment of an autonomous venipuncture device of the present invention is shown in FIG. 3. Autonomous venipuncture device 200 is designed such that a non-trained patient can use device 200 on themselves without the need for any assistance by a trained professional. Autonomous venipuncture device 200 will enable a patient to provide themselves with therapy or to draw their own blood for lab testing in the comfort of home. Autonomous venipuncture device 200 includes a base 201 which in use will sit flat against an arm A of a patient. Although FIG. 3 shows an arm A or a human patient, autonomous venipuncture device 200 can be secured to any limb or any mammal. Base 201 includes a front set of apertures 202 and a rear set of apertures 203 which carry a front strap 204 and a rear strap 205, respectively. Front strap 204 and rear strap 205 are utilized to secure autonomous venipuncture device 200 around arm A of a patient. Front strap 204 and rear strap 205 can be any strap capable of securing autonomous venipuncture device 200 around arm A of a patient. For example, straps 204 and 205 can include a buckle system, a hook and loop system, or an automatic mechanism in order to secure the device 200 around arm A. Base 201 will also include an insertion site aperture 206. As will be discussed in greater detail below, the venipuncture tool 207 will go through insertion site aperture 206 of base 201 and into arm A of the patient once the venipuncture tool 207 has been placed in the proper location.

In one or more embodiments of the present invention, the venipuncture tool 207 can be selected from the group consisting of a needle, a butterfly needle, an intravenous catheter, a peripherally inserted central catheter, and/or a midline catheter.

Although not shown in the drawings, autonomous venipuncture device 200 is powered by a battery. Autonomous venipuncture device 200 also includes an internal processor 227 in order to analyze the data it collects and determine, using a combination of algorithms and machine learning, where to place the venipuncture tool 207 within arm A of the user.

Autonomous venipuncture device 200 is placed in the proper location with ultrasonic technology and optionally with infrared technology. Ultrasonic device 208 takes an ultrasonic image of the inside of arm A of the patient which provides data on both the position and depth of a vein in the arm A of the patient. In one or more embodiments, the ultrasonic device is a portable handheld linear ultrasound such as those made by SonoQue™. Although not shown in the drawings, it is contemplated that autonomous venipuncture device 200 includes a mechanism to apply ultrasound gel to arm A of the patient for the ultrasonic device 208 to take a clearer ultrasonic image of the inside of arm A of the patient. In some embodiments of the present invention, an infrared camera 209 takes an image of the arm A of the patient to provide additional information on the position of the vein in the arm A of the patient.

Once the data from the ultrasonic device 208 and optionally the infrared camera 209 has been collected, the data is sent to the internal microprocessor or computer to determine the optimal position to insert the venipuncture tool 207 into arm A of the patient. The venipuncture tool 207 is carried by a venipuncture tool holder 210. Venipuncture tool holder 210 is moveable along both the X-axis and the Y-axis, with the X-axis being defined for the purposes of this application as moving the venipuncture tool holder 210 and the venipuncture tool 207 left and right alongside arm A and the Y-axis being defined for the purposes of this application as moving the venipuncture tool holder 210 and the venipuncture tool 207 into and out of arm A.

An X-axis motor 211 moves an X-linear motion stage 212 along an X-axis screw 213 and X-axis rail 229 in order to move the venipuncture tool holder 210 and the venipuncture tool 207 in the X-axis direction. Specifically, threads of the X-axis screw 213 interact with threads of a nut positioned within an aperture of the X-linear motion stage 212 such that rotation in a first direction moves the X-linear motion stage 212 in a right-facing direction and rotation in a second direction move the X-linear motion stage 212 in a left-facing direction. X-linear motion stage 212 also moves along X-axis rail 229 in a similar manner. While a Y-axis motor 214 moves a Y-linear motion stage 215 along a Y-axis rail 216 in order to move the venipuncture tool holder 210 and the venipuncture tool 207 in the Y-axis direction. Specifically, a Y-axis screw (not shown) interacts with threads of a nut (not shown) positioned within an aperture of the Y-linear motion stage 215 such that rotation in a first direction moves the Y-linear stage 215 in an inside limb direction and rotation in a second direction move the Y-linear stage 215 in an outside arm direction.

Therefore, once the internal microprocessor or computer has collected and analyzed the data collected from the ultrasonic device 208 and optionally the infrared camera 209, the internal microprocessor or computer will then direct the X-axis motor 211 to move the X-linear motion stage 212 along X-axis screw 213 and the Y-axis motor 214 to move the Y-linear motion stage 215 along Y-axis rail 216 so as to properly position the venipuncture tool holder 210 so that the venipuncture tool 207 can be accurately inserted into a vein in arm A of the patient.

The autonomous venipuncture device 200 also contains an end-stop switch for each motor 211 and 214. As shown in FIG. 3, only end-stop switch 217 which works in conjunction with motor 211 is visible. Each end-stop switch makes sure that the position of the venipuncture tool 207 is always at a zero position prior to use. This is needed to make sure that the internal microprocessor or computer is always determining the proper position for insertion of the venipuncture tool from the same zero position. Such that when the collected data from the ultrasonic device 208 and optionally the infrared camera 209 is processed by the internal microprocessor or computer, it is always working from the venipuncture tool 207 being in the same starting position.

In another embodiment of the present invention, the autonomous venipuncture device 200 would have a means to connect to the internet 228. Internet access will allow the autonomous venipuncture device 200 to be remotely operated by a trained professional P. The trained professional will only need a user interface, such as a joystick J as shown in FIG. 3, on their end to control the autonomous venipuncture device 200. The ultrasonic device 208 and optionally the infrared camera 209 will still collect data on the position and depth of a vein in the arm A of the patient. However, that data will then be displayed to the trained professional in real time who will then send controls to the X-axis motor 211 to move the X-linear motion stage 212 along X-axis screw 213 and the Y-axis motor 214 to move the Y-linear motion stage 215 along Y-axis rail 216 so as to properly position the venipuncture tool holder 210 so that the venipuncture tool 207 can be accurately inserted into a vein in arm A of the patient.

The embodiment of the autonomous venipuncture device 200 as shown in FIG. 3 includes a drug delivery system that can administer drugs through the venipuncture tool 207 once said tool has been placed in a vein in arm A of the patient. Venipuncture tool holder 210 has a pipe access port 218 that gives access to an end of the venipuncture tool 207. A pipe 219 can be inserted into the port 218 with the other end of pipe 219 being connected to a drug/fluid storage device 220. Drug/fluid storage device 220 can be selected from the group consisting of a syringe tube or a fluid bag. FIG. 3 shows a syringe tube 220. Drug/fluid storage device 220 can contain any drug or fluid needed for a particular situation. For example, the drug contained within drug/fluid storage device 220 can be selected from any recommended or prescribed drug and the fluid can be any medication needed to be used for intravenous infusion on the patient. It is also contemplated that the drug/fluid storage device 220 could be empty prior to being utilized to store blood drawn from arm A of the patient.

Autonomous venipuncture device 200 utilizes a separate syringe motor 221 which moves the plunger plate 222 up and down along syringe screw 223 in order to operate the syringe 226 connected to the plunger plate 222. The operation of syringe 226 can be utilized to inject a drug or fluid stored in drug/fluid storage device 220 through the pipe 219 and into the venipuncture tool 207 or to draw blood from arm A through venipuncture tool 207 to be stored in drug/fluid storage device 220. Although not shown in the drawings, autonomous venipuncture device 200 may be equipped with more than one drug/fluid storage device 220 in order to deliver more than one drug into arm A of the patient or to be able to both deliver a drug into arm A of the patient while also being able to draw blood from the patient or to deliver two different drugs into arm A of the patient.

Although not shown in the drawings, it is contemplated that autonomous venipuncture device 200 could also include a mechanism to sterilize arm A of the patient prior to insertion of the venipuncture tool 207. Although not shown in the drawings, it is also contemplated that autonomous venipuncture device 200 could also include a mechanism to automatically close the insertion site created by insertion of the venipuncture tool 207 into arm A of the patient.

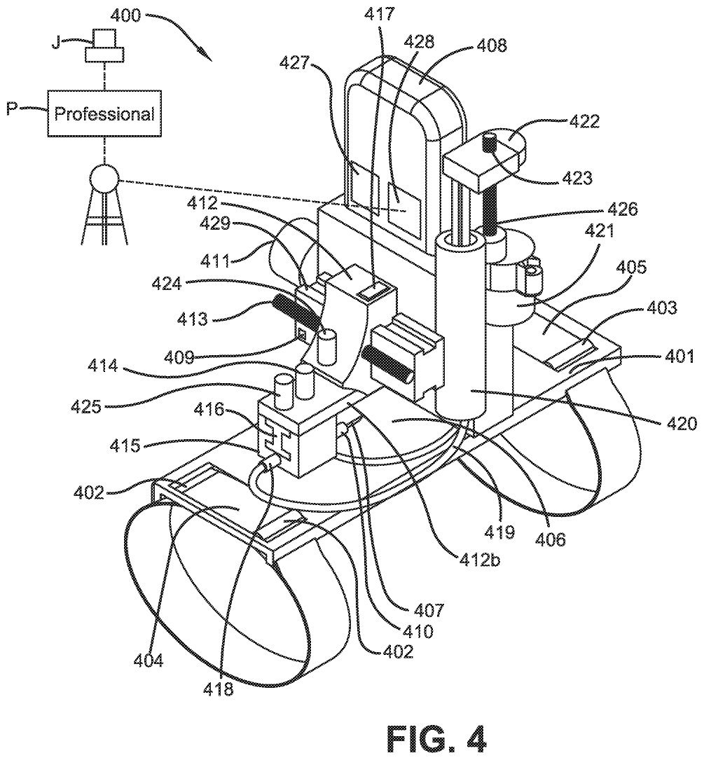

An additional embodiment of an autonomous venipuncture device of the present invention is shown in FIG. 4. Autonomous venipuncture device 400 includes a base 401 which in use will sit flat against a limb of a patient (not shown). Base 401 includes a front set of apertures 402 and a rear set of apertures 403 which carry a front strap 404 and a rear strap 405, respectively. Front strap 404 and rear strap 405 are utilized to secure autonomous venipuncture device 400 around a limb of a patient. Base 401 will also include an insertion site aperture 406. As will be discussed in greater detail below, the venipuncture tool 407 will go through insertion site aperture 406 of base 401 and into the limb of the patient once the venipuncture tool 407 has been placed in the proper location.

Although not shown in the drawings, autonomous venipuncture device 400 is powered by a battery. Autonomous venipuncture device 400 also includes an internal processor 427 in order to analyze data it collects and determine, using a combination of algorithms and machine learning, where to place the venipuncture tool 407 within the limb of the user. Autonomous venipuncture device 400 is placed in the proper location with ultrasonic technology and optionally infrared technology. Ultrasonic device 408 takes an ultrasonic image of the inside of the limb of the patient which provides data on both the position and depth of a vein in the limb of the patient. In some embodiments of the present invention, an infrared camera 409 takes an image of the limb of the patient to provide additional information on the position of the vein in the limb of the patient.

Once the data from the ultrasonic device 408 and optionally the infrared camera 409 has been collected, the data is sent to the internal microprocessor or computer to determine the optimal position to insert the venipuncture tool 407 into the limb of the patient. The venipuncture tool 407 is carried by a venipuncture tool holder 410. Venipuncture tool holder 410 is moveable along both the X-axis and the Y-axis, with the X-axis being defined for the purposes of this application as moving the venipuncture tool holder 410 and the venipuncture tool 407 left and right alongside the limb and the Y-axis being defined for the purposes of this application as moving the venipuncture tool holder 410 and the venipuncture tool 407 into and out of the limb.

An X-axis motor 411 moves an X-linear motion stage 412a along an X-axis screw 413 and an X-axis rail 429 in order to move the venipuncture tool holder 410 and the venipuncture tool 407 in the X-axis direction. While a Y-axis motor 414 moves a Y-linear motion stage 415 along a Y-axis rail 416 in order to move the venipuncture tool holder 410 and the venipuncture tool 407 in the Y-axis direction.

Autonomous venipuncture device 400 additionally includes a Z-axis motor 424 and a Y-angle motor 425. Z-axis motor 424 moves Z-axis stage 412b up and down in a direction perpendicular the limb of the patient while Y-angle motor 425 spins Y-linear motion stage 415 to align the venipuncture tool 407 along the direction of the vein in the limb of the patient.

Therefore, once the internal microprocessor or computer has collected and analyzed the data collected from the ultrasonic device 408 and optionally the infrared camera 409, the internal microprocessor or computer will then direct the X-axis motor 411 to move the X-linear motion stage 412 along X-axis screw 413, the Z-axis motor 424 to move the Z-axis stage 412b up and down, the Y-angle motor 425 to spin the Y-linear motion stage 415, and the Y-axis motor 414 to move the Y-linear motion stage 415 along Y-axis rail 416 so as to properly position the venipuncture tool holder 410 so that the venipuncture tool 407 can be accurately inserted into a vein in a limb of the patient.

The autonomous venipuncture device 400 also contains an end-stop switch for each motor 411, 414, 424, and 425. As shown in FIG. 4, only end-stop switch 417 which works in conjunction with motor 411 is visible. Each end-stop switch makes sure that the position of the venipuncture tool 407 is always at a zero position prior to use. This is needed to make sure that the internal microprocessor or computer is always determining the proper position for insertion of the venipuncture tool from the same zero position. Such that when the collected data from the ultrasonic device 408 and optionally the infrared camera 409 is processed by the internal microprocessor or computer, it is always working from the venipuncture tool 407 being in the same starting position.

The embodiment of the autonomous venipuncture device 400 as shown in FIG. 4 can administer drugs through the venipuncture tool 407 once said tool has been placed in a vein in a limb of the patient. Venipuncture tool holder 410 has a pipe access port 418 that gives access to the venipuncture tool 407. A pipe 419 can be inserted into the port 418 with the other end of pipe 419 being connected to a drug/fluid storage device 420. Drug/fluid storage device 420 can be selected from the group consisting of a syringe tube or a fluid bag. FIG. 4 shows a syringe tube 420. Drug/fluid storage device 420 can contain any drug needed for a particular situation. For example, the drug contained within drug/fluid storage device 420 can be selected from any recommended or prescribed drug. It is also contemplated that the drug/fluid storage device 420 could be utilized to store blood drawn from a limb of the patient.

Autonomous venipuncture device 400 utilizes a separate syringe motor 421 which moves the plunger plate 422 up and down along syringe screw 423 in order to operate the syringe 426 connected to the plunger plate 422. The operation of syringe 436 can be utilized to inject a drug or fluid stored in drug/fluid storage device 420 through the pipe 419 and into the venipuncture tool 407 or to draw blood from arm A through venipuncture tool 407 to be stored in drug/fluid storage device 220. Although not shown in the drawings, autonomous venipuncture device 400 may be equipped with more than one drug/fluid storage device 420 in order to deliver more than one drug into a limb of the patient or to be able to both draw blood and also deliver a drug into the limb of the patient.

In light of the foregoing, it should be appreciated that the present invention significantly advances the art by providing a portable autonomous venipuncture device and method of its use that is structurally and functionally improved in several ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A portable autonomous venipuncture device to be secured to a limb of a patient comprising:
a. a limb-mountable base configured to be mounted to the limb of a patient;
b. a venipuncture tool holder carried on said base
c. a venipuncture tool carried by said venipuncture tool holder and having an insertion tip;
d. an ultrasonic device carried on said base; and
e. a processor,
wherein said ultrasonic device is configured to provide an ultrasonic image of inside of the limb of the patient to provide location data on the position and depth of a vein in the limb, and wherein said processor is configured to use the location data to move the insertion tip of the venipuncture tool into the vein of the patient.

2. The portable autonomous venipuncture device of claim 1, further comprising a first motor to control movement of said venipuncture tool holder along an X-axis and a second motor to control movement of said venipuncture tool holder along a Y-axis.

3. The portable autonomous venipuncture device of claim 4, wherein said processor directs the first motor to move the X-linear motion stage along the X-axis rail to place said venipuncture tool holder into an X-axis position; and wherein said processor directs the second motor to move the Y-linear motion stage along the Y-axis rail to place said venipuncture tool holder into a Y-axis position.

4. The portable autonomous venipuncture device of claim 1, further comprising an infrared camera configured to provide an infrared image of the limb of the patient to provide additional location data on the position of the vein in the limb, and wherein said processor is configured to use the location data from the ultrasonic device said additional location data from said infrared camera to move the insertion tip of the venipuncture tool into the vein of the patient.

5. The portable autonomous venipuncture device of claim 1, wherein said base further comprises a front pair of apertures, a rear set of apertures, and an insertion site aperture wherein said insertion site aperture gives said venipuncture tool access to said limb through said base.

6. The portable autonomous venipuncture device of claim 6, further comprising a front strap placed through said front pair of apertures and a rear strap placed through said rear set of apertures.

7. The portable autonomous venipuncture delivery device of claim 1, further including a drug delivery system carried on said base, wherein said drug delivery system comprises:
a. a drug/fluid storage device carried on the limb-mountable base;
b. a pipe connected to said drug/fluid storage device;
c. a syringe motor carried on the limb-mountable base;
d. a syringe screw;
e. a plunger plate connected to the syringe screw; and
f. a syringe connected to the plunger plate and carried by the drug/fluid storage device; and
wherein the syringe motor controls movement of the plunger plate along the syringe screw to operate the syringe to deliver a drug carried within the drug/fluid storage device through the pipe and into the limb or to operate the syringe to draw blood from the limb and deliver the drawn blood through the pipe to the drug/fluid storage device.

8. The portable autonomous venipuncture delivery device of claim 8, wherein the venipuncture tool holder further includes a pipe access port, and wherein a first end of the pipe is connected to the drug/fluid storage device and a second end of the pipe is connected to the pipe access port.

9. A medical assistance and drug delivery device comprising a medical assistance device and an autonomous venipuncture device including a processor, wherein the medical assistance device provides medical assistance to a patient while also producing a medical data set, wherein the processor assesses the medical data set to determine if an injection-treatable condition is present, and if the injection-treatable condition is present, the autonomous venipuncture device delivers an injection.

10. A method of treating an arrhythmia of a patient comprising the steps of:
a. placing electrode pads of an automated external defibrillator (AED) device on a patient's chest;
b. placing an autonomous venipuncture device on a limb of the patient, the autonomous venipuncture device having a venipuncture tool having an insertion tip;
c. reading by the AED device of a heart rhythm of the patient to determine if said rhythm is a shockable arrhythmia;
d. optionally delivering at least one shock to the chest of the patient through the pair of electrode pads;
e. determining if drug assistance is needed to treat the heart rhythm;
f. determining an insertion position on the limb utilizing the autonomous venipuncture device;
g. inserting, if drug assistance was determined to be needed, the insertion tip of the venipuncture tool into the insertion position on the limb of the patient; and
h. delivering drug assistance to the patient through the venipuncture tool.

11. The method of claim 10, wherein the drug assistance can be delivered through the venipuncture tool directly by the autonomous venipuncture device or by a trained professional.

12. The method of claim 11, wherein the drug assistance is delivered directly by the autonomous venipuncture device by a syringe motor of the autonomous venipuncture device operating a syringe connected to a plunger plate of the autonomous venipuncture device along a syringe screw of the autonomous venipuncture device to deliver a drug carried within a drug/fluid storage device of the autonomous venipuncture device through a pipe that connects to the venipuncture tool.

13. The method of claim 10, wherein the determining of the insertion position is conducted by an ultrasonic device of the autonomous venipuncture device taking an ultrasonic image of inside of the limb of the patient to provide location data on the position and depth of a vein in the limb, and wherein said processor is configured to use the location data to move the insertion tip of the venipuncture tool into the vein of the patient.

14. The method of claim 14, wherein a first motor of the autonomous venipuncture device controls the positioning of said venipuncture tool along an X-axis and a second motor of the autonomous venipuncture device controls positioning of said venipuncture tool along a Y-axis.

15. The method of claim 14, wherein the determining of the insertion position is further conducted by an infrared camera of the autonomous venipuncture device which takes an infrared image of the limb of the patient to provide additional data on the position of the vein in the limb, and wherein said processor takes said data from said ultrasonic device and said additional data from said infrared camera to move the insertion tip of the venipuncture tool into the insertion position.
